(19) United States
(12) Patent Application Publication
(10) Pub. No.: US 2019/0108912 A1
(43) Pub. Date: Apr. 11, 2019

(54) METHODS FOR PREDICTING OR DETECTING DISEASE

(71) Applicant: IQUITY, INC., Nashville, TN (US)

(72) Inventors: Charles F. Spurlock, III, Nashville, TN (US); Julia B. Polk, Nashville, TN (US)

(21) Appl. No.: 16/115,444

(22) Filed: Aug. 28, 2018

Related U.S. Application Data
(60) Provisional application No. 62/568,739, filed on Oct. 5, 2017.

(51) Int. Cl. G16H 50/20; G16H 50/70; G16H 50/30; G06F 19/18; G06F 15/18

(52) U.S. Cl. CPC G16H 50/20; G16H 50/70; G06F 15/18; G06F 19/18; G16H 50/30

(57) ABSTRACT

The invention provides methods that use machine learning to discover within clinical data patterns that are predictive of disease. Clinical data from across a population is provided as input to a machine learning system. The autonomous machine learning system discovers associations in data from a plurality of data sources obtained from a population and correlates the associations to health status of patients in the population. The methods may further include providing patient data from an individual; and predicting, by the machine learning system, a health state for the individual when the patient data presents one or more of the discovered associations.

### TECHNICAL FIELD

The invention relates to predicting disease.

### BACKGROUND

The early detection of disease allows for early intervention. For many diseases, early detection increases the likelihood of a successful treatment and gives the patient the best range of options for quality-of-life decisions. Unfortunately, detecting a disease has historically followed a pattern in which a patient seeks help from a medical provider only after experiencing symptoms that affect the patient's quality of life. Thus, even though the scientific understanding of disease is always expanding, there continue to be cases in which a disease will advance significantly in a patient before it is detected.

Due to the reliance on patient-initiated subjective complaints, a physician's subjective analysis, and the subsequent diagnoses of exclusion (i.e., diagnosing by process of elimination), many patients go undiagnosed, or are misdiagnosed, which unfortunately leads to delayed treatment and poor outcomes. This is especially true in the fields of inflammatory disease, cancer, and autoimmune disease. In particular, for such diseases in which tissue degeneration and loss of function is cumulative, early detection and treatment would provide significant benefits in mitigating damage, reducing flare-ups, and improving overall recovery. Unfortunately, in many case, patients begin treatment late enough that they do not reap the potential benefits offered by early disease detection and diagnosis.

Even once a patient is properly diagnosed with a disease, it is sometimes difficult to assess the severity of the disease, and therefore difficult to provide an accurate prognosis of disease to the patient. Predicting the course, or progression, of a disease in a particular patient is complicated by many parallel competing factors, such as environmental conditions, genetics, severity and impact of the symptoms, all of which vary from patient to patient. In addition, the prediction of disease course or severity is mostly subjective because it is assessed using a physician's general medical knowledge regarding disease outcome and the interrelation of diseases. As such, this type of subjective assessment varies from clinician to clinician.

Furthermore, post-diagnosis, physicians are often faced with patients who do not follow the “normal” prognosis of the diagnosed disease and are suddenly trending towards a more negative prognosis, at which point, it might be too late to reverse any adverse effects with a revised treatment plan. Predicting adverse events, relapses and flare-ups related to diseases is difficult if not impossible with the current state of technology. Additionally, a patient's treatment compliance is difficult to monitor and predict, so it is often difficult to provide the appropriate treatment regimen to a patient.

There is a need for better healthcare prediction and management. Specifically, there is a need for a system that continuously accumulates health related data from various sources and analyzes such data to assist clinicians in making timely and accurate diagnoses, assessing outcomes, stratifying the severity of a disease, predicting treatment compliance, and providing a patient with a prognosis of disease, so as to properly counsel and treat a patient.

### SUMMARY

The invention provides methods that use machine learning to discover within clinical data patterns that are predictive of disease. Clinical data from across a population is provided as input to a machine learning system. That clinical data may include such data types as medical records, claims data, and test results, among others. The machine learning system processes the clinical data and discovers latent patterns that are predictive of disease. Because the data include medical events from the population over time, including patterns of classification codes, test results, and disease outcomes, the machine learning system can discover sequences and combinations of events that would not be apparent to a human reviewer but that are nevertheless reliable predictors of medically-important outcomes. Moreover, the machine learning system can correlate certain combinations of events with future outcomes. In an example, the system may learn that if certain classification codes are associated with each other within a few years for a patient, then that patient has a high probability of a future diagnosis for a certain disease. After repeatedly finding that association between those data entries (the classification codes) across the population, the machine learning system learns the association and its correlation to the future diagnosis. The system is robust in that it can learn any arbitrary number of patterns or associations across the population data and it is free from a priori expectations that a health professional may have in mind. The machine learning system can discover associations over any span of time, without bias, and reliably build the correlations between those associations and future disease states. Furthermore, the system can differentiate between associations related to early-onset forms of a disease (e.g., juvenile forms) and late-onset forms of a disease (e.g., adult forms).

Due to the ability of the machine learning system to discover within clinical data associations among events that correlate to future disease outcomes, the system is useful in predicting health status for individuals. The operating machine learning system may have access to clinical data for an individual and, as new data is added, the system may detect disease-correlated patterns. In fact, the system may detect patterns that have already been correlated, by the system, to specific disease events at predictable times in the future. For example, a patient may complain to her doctor of restless legs. A year later, that patient may report feeling a widespread dull ache and chronic fatigue. Even if the second report is with a different doctor or at a different clinic, those symptoms may be entered into medical records according to classification of disease codes. The machine learning system may recognize that those codes, when associated in that pattern, have consistently been predictive of a diagnosis, 3 to 5 years in the future, of fibromyalgia. Upon detecting that association among the data for the individual, the machine learning system provides a report to a health professional that predicts a risk of fibromyalgia for this patient in the future. This predictive report allows the health professional to initiate additional tests and begin treatment interventions far earlier than would otherwise have been possible.

In certain other aspects, systems of the invention detect patterns of comorbidities that have already been correlated, by the system, to specific disease events at predictable times in the future. For example, a patient may report chronic fatigue and a rash to her doctor. That doctor may or may not diagnosis her with lupus. That same patient may then complain a year later to her doctor of restless legs. And two years later, that same patient may report feeling a widespread dull ache. Even if all of the reports are provided to different doctors or at a different clinic, those symptoms (with or without a first diagnosis) may be entered into medical records according to classification of disease codes. The machine learning system recognizes that those codes, when associated in that pattern, have consistently been predictive of a first diagnosis of lupus and second concurrent diagnosis, years in the future, of fibromyalgia. Upon detecting that association among the data for the individual, the machine learning system provides a report to a health professional that predicts a risk of lupus and fibromyalgia for this patient in the future. This predictive report allows the health professional to initiate additional tests and begin treatment interventions far earlier than would otherwise have been possible, including the prediction of comorbidities.

In another aspect, machine learning systems of the invention predict patient readmission rates. In this regard, systems of the invention identify trends that indicate a hospital readmission is likely. Having that information influences patient care management post-release in order to minimize the chance of readmission. Certain principal diagnoses are correlated with the possibility of readmission and are preferably weighted. The aggregate of the principal diagnosis scores are then used to obtain a likelihood of readmission post-release. Factors useful in the algorithm include, but are not limited to, schizophrenia, alcohol-related disorders, congestive heart failure, heart valve disorders, hypertension (with or without complications and respiratory failure), respiratory failure, anemia, systemic lupus erythematosus, and other chronic conditions. Additional factors include age, insurance status, and the combination of different conditions. The table below (Table 1) is a non-exhaustive list of certain exemplary indications and their associated rate of readmission after 7 and/or 30 days post-release.

TABLE 1
Exemplary Indications

| | Percent Expected | Percent Expected |
|---|---|---|
| | Readmission After 7 | Readmission After 30 |
| Condition | Days | Days |
| Psychotic disorders | 9% | 22.9% |
| Congestive Heart Failure | 7.4% | 23.2% |
| Alcohol-Related disease | 7.5% | 21.5% |
| Lupus | — | 16.5% |
| All Chronic Conditions | — | 2.7%-18.6% |
| | | (depending on number |
| | | of conditions) |
| Anemia | — | 21.2% |

Operation of the machine learning system may be integrated with clinical services laboratories in that the system can use data from multiple sources and of multiple data types including laboratory assay results. The system can use medical records, claims data, and results from clinical assays. Thus, the results from laboratory tests, such as sequencing, expression profiling, blood tests, or the like can be provided to the machine learning system as part of the clinical data, along with medical records or claims data, and the machine learning system can provide predictions and results in response to all such inputs.

Thus, the system provides the ability to predict and detect future diseases and comorbidities. The early prediction and detection gives health professionals the ability to begin additional testing, to test for conditions that they may not otherwise have been alerted to, and to initiate early treatment. The system operates without bias or undue emphasis on any single office visit, checkup, or test result. By giving health professionals the ability to predict and detect disease very early, methods of the invention allow for early intervention and thus provide for the best possible clinical outcomes. The opportunity for such early intervention is valuable for degenerative diseases that have historically defied early detection. Thus, methods of the invention may have particular value in the treatment of degenerative diseases or conditions such as multiple sclerosis, irritable bowel syndrome, Crohn's disease, ulcerative colitis, amyotrophic lateral sclerosis, fibromyalgia, rheumatoid arthritis, or lupus. By early detection and intervention, the effects of such diseases may be minimized, and for a great number of people, life can be extended and quality of life can be greatly improved.

In certain aspects, the invention provides methods of predicting health status. Methods include discovering via an autonomous machine learning system associations in data from a plurality of data sources obtained from a population and correlating the associations to health status of patients in the population. The data may be formatted such that each entry in the data is specific to a patient from the population and assigned to a pre-defined category. Discovering an association may include observing, in a plurality of patients, co-occurrences of event categories significantly different from an expected number of co-occurrences. The method may further include adding the discovered associations into the data as events and continuing to discover associations in the data that includes the initially-discovered associations. In some embodiments, the methods include providing patient data from an individual and predicting, by the machine learning system, a health state for the individual when the patient data presents one or more of the discovered associations.

In certain embodiments, the methods include obtaining a sample from the individual, performing an assay on the sample to produce clinical results, and including the clinical results in the patient data from the individual. For example, a sample comprising nucleic acid may be obtained from the individual and the assay may include sequencing the nucleic acid, such that the clinical results include sequences or expression level. Providing the patient data may include obtaining clinical diagnostic codes for the individual.

The plurality of data sources may include one or more of claims data, demographic data, geographic data, medical history, genetic data, and laboratory test results. Any suitable machine learning system may be used including, for example, any one or more of a random forest, a grid search, a support vector machine, and a neural network. In preferred embodiments, the autonomous machine learning system comprises a random forest with hyperparameters that have been optimized by grid search. Preferably, the autonomous machine learning system discovers the associations via operations that include at least a period of unsupervised learning.

In certain embodiments, the discovered associations including patterns of association between claims data and at least one other data source (such as, for example, RNA expression information or genomic expression information). In such embodiments, the method preferably includes providing patient data from an individual and predicting, by the machine learning system, a health state for the individual when the patient data presents one or more of the discovered associations. In some embodiments, the patient data presents a discovered association between claims data and RNA expression information and the predicted health state for the individual includes a predicted onset of a disease such as multiple sclerosis, irritable bowel syndrome, Crohn's disease, ulcerative colitis, fibromyalgia, rheumatoid arthritis, or lupus.

In some embodiments, the discovered associations include a patient-specific pattern occurring within claims data, and a recurrence of the patient-specific pattern within the claims data is correlated to a later onset of a disease, or the reoccurrence or relapse of disease, such as an inflammatory or neurodegenerative disease. The patient-specific pattern may include combinations of diagnostic codes reported over time that are predictive of the disease. Other aspects of the invention provide methods to identify the minimum number of inputs required to establish the presence of a disease. In certain embodiments, a set of parameters may be identified as being suspected to be related to diagnosis of a disease using one or more machine learning systems and members of the set may then be refined using a different machine learning system. Accordingly, some of the training steps may be unsupervised using unlabeled data while subsequent training steps (e.g., member refinement) may use supervised training techniques such as regression analysis using the set of parameters autonomously identified by the first machine learning system. Importantly, the machine learning system progressively eliminates members of the set to a point at which elimination of further members of the set fails to increase the sensitivity and/or specificity of diagnosis of the disease. In some embodiments, the machine learning algorithm identifies the minimum number of inputs required to establish the presence of the disease. The machine learning system may include a neural network, a random forest, grid search, Bayesian classifier, logistic regression, decision tree, gradient-boosted tree, multilayer perceptron, one-vs-rest, and Naive Bayes, cluster analysis, a support vector machine (SVM), or a boosting algorithm. In some embodiments, the machine learning algorithm includes a random forest comprising a plurality of decision trees. The decision trees receive parameters such as: ICD codes; CPT codes; HCPCS codes; patient demographic data; and patient geographic data.

In any of the embodiments herein, the disease may be a chronic inflammatory disease. The inflammatory disease may be atherosclerosis, stroke, asthma, uveitis, sinusitis, angioedema, psoriasis, psoriatic arthritis, multiple sclerosis, and Alzheimer's disease. The disease may be an autoimmune disease such as fibromyalgia, rheumatoid arthritis, lupus, ankylosing spondylitis, Hashimoto's thyroiditis, Sjögren's syndrome, Graves' disease, inflammatory bowel disease, Crohn's disease, ulcerative colitis, celiac disease, pernicious anemia, and sinusitis. In other embodiments, the disease may be more than one disease occurring at the same time resulting in comorbidity. In yet another embodiment, onset of the comorbidity of diseases may occur at a point in time after the first diagnosis of a disease. Preferably, the machine learning algorithm is implemented in a computing system comprising at least one processor coupled to a tangible, non-transitory memory subsystem. In certain embodiments, the machine learning algorithm includes a neural network. Methods of the invention may further be used for determining disease outcome of a patient includes stratifying the data to identify shared commonalties amongst the data. The shared commonalties are used to generate a disease specific network. In other embodiments, the disease networks may be used to construct data sets which can be used to train models. In some embodiments, the machine learning algorithm identifies patterns within training data sets. Data sets used as training data include outcome data, phenotypic data, environmental data, demographic data, geographic data, genetic data, clinical data, insurance claim data, and treatment data. The models can then be used to identify patients with an increased likelihood of having or developing a disease. The models can be used to differentiate between associations related to early-onset forms of a disease (e.g., juvenile forms) and late-onset forms of a disease (e.g., adult forms). The machine learning system may identify the outcome of a patient as related to the identified disease, well before the risk of disease would be discovered by a patient him- or herself, or in the course of routine doctor visits. The outcome identified by the machine learning algorithm may be diagnosis, comorbidity, severity, prognosis, treatment selection, treatment compliance, reoccurrence, mortality, effectiveness of treatment or quality of life.

### BRIEF DESCRIPTION OF THE DRAWINGS

FIG. 1 diagrams a method of predicting health status.

FIG. 2 shows a machine learning system according to certain embodiments.

FIG. 3 shows a machine learning system discovering associations in the data.

FIG. 4 diagrams a system for predicting health status by method of the invention.

FIG. 5 shows a report provided by systems and methods of the invention.

### DETAILED DESCRIPTION

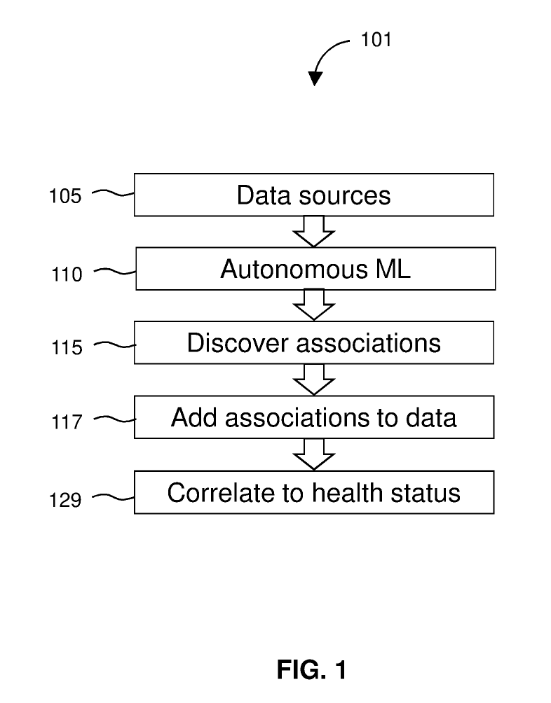

FIG. 1 diagrams a method 101 of predicting health status. The method 101 includes accessing 105 multiple data sources of clinical data from a population and operating 110 an autonomous machine learning system. The autonomous machine learning system discovers 115 associations in the clinical data from the plurality of data sources from the population. In an optional embodiment, those discovered associations are added 117 back into the data as data entries themselves. Additionally, the machine learning system correlates 129 the associations to health status of patients in the population.

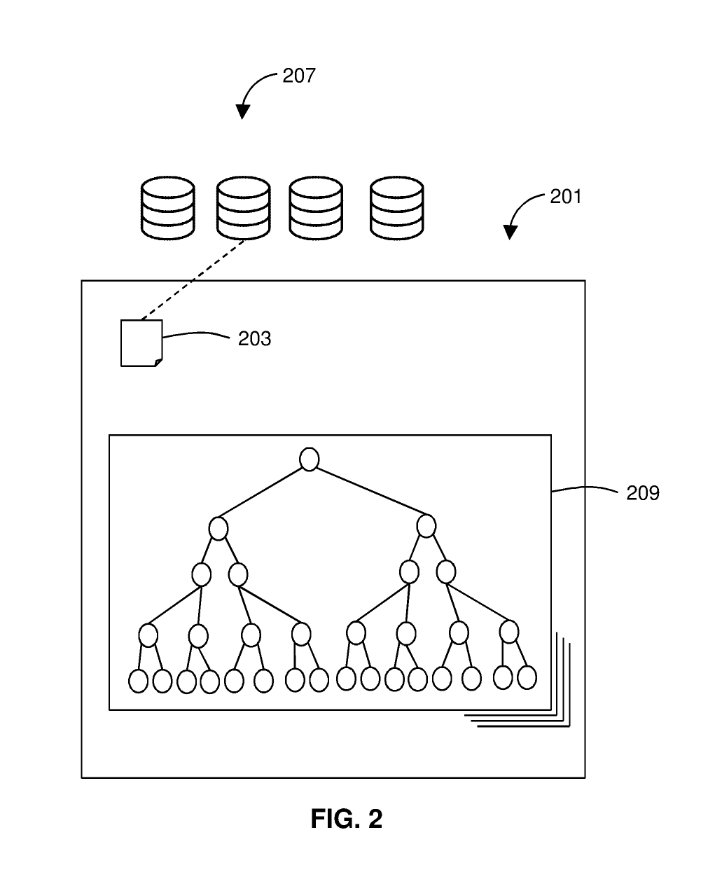

FIG. 2 shows a machine learning system 201 according to certain embodiments. The machine learning system 201 accesses data from a plurality of sources 207. Any suitable source of clinical data 207 may be provided 105 to the machine learning system 201. Generally, clinical data includes data that is collected during the course of ongoing patient care or as part of a formal clinical trial program. Types of clinical data include health records/medical records, administrative data, claims data, patient or disease registries, health surveys, clinical trial data, and test results such as clinical laboratory assay results.

Health records, or medical records, generally include electronic clinical data which is obtained at the point of care at a medical facility, hospital, clinic or practice. Often referred to as the electronic medical record (EMR), the EMR typically includes administrative and demographic information, diagnosis, treatment, prescription drugs, laboratory tests, physiologic monitoring data, hospitalization, patient insurance, etc. Sources of EMR include individual organizations such as hospitals or health systems. EMR may be accessed through larger collaborations, such as the NIH Collaborator Distributed Research Network, which provides mediated or collaborative access to clinical data repositories by eligible researchers. Additionally, the UW De-identified Clinical Data Repository (DCDR) and the Stanford Center for Clinical Informatics allow for initial cohort identification.

Administrative data, often associated with electronic health records, generally includes hospital discharge data reported to a government agency like AHRQ, or data from the Healthcare Cost & Utilization Project (H-CUP). H-CUP is a free, on-line query system based on data from the Healthcare Cost and Utilization Project (HCUP). It provides access to health statistics and information on hospital inpatient and emergency department utilization.

Claims data include the billable interactions (insurance claims) between insured patients and the healthcare delivery system. Claims data falls into four general categories: inpatient, outpatient, pharmacy, and enrollment. The sources of claims data can be obtained from the government (e.g., Medicare) and/or commercial health firms (e.g., United HealthCare). Claims data may be accessed as Basic Stand Alone (BSA) Medicare Claims Public Use Files (PUFs), a claim-level file in which each record is a claim incurred by a 5% sample of Medicare beneficiaries. Claims include inpatient/outpatient care, prescription drugs, DME, SNF, hospice, etc. Additionally, Medicaid data may be accessed via a service such as the Medicaid Analytic eXtract data, which contains state-submitted data on Medicaid eligibility, service utilization and payments. The CMS-64 provides data on Medicaid and SCHIP Budget and Expenditure Systems.

Disease registries are clinical information systems that track a narrow range of key data for certain chronic conditions such as Alzheimer's Disease, cancer, diabetes, heart disease, and asthma. Registries often provide critical information for managing patient conditions. Disease registries include, for example, the Global Alzheimer's Association Interactive Network (GAAIN), National Cardiovascular Data Registry (NCDR), National Program of Cancer Registries, The National Trauma Data Bank (NTDB), and the Surveillance, Prevention, and Management of Diabetes Mellitus Datalink (SUPREME DM).

Health surveys generally include government or industry sponsored evaluations of population health. These surveys of the most common chronic conditions are generally conducted to provide prevalence estimates. National surveys are one of the few types of data collected specifically for research purposes, thus making it more widely accessible. Examples include the Medicare Current Beneficiary Survey, National Health & Nutrition Examination Survey (NHANES), The Medical Expenditure Panel Survey (MEPS), the National Center for Health Statistics, Center for Medicare & Medicaid Services Data Navigator, and the National Health and Aging Trends Study (NHATS).

Clinical data may also be obtained from clinical trials registries and databases such as ClinicalTrials.gov, WHO International Clinical Trials Registry Platform (ICTRP), the European Union Clinical Trials Database, the ISRCTN Registry (BioMed Central), or CenterWatch.

In preferred embodiments, the plurality of data sources 207 feed into the machine learning system 201. Any suitable machine learning system 201 may be used. For example, the machine learning system 201 may include one or more of a random forest, a support vector machine, and a neural network. In the depicted embodiment, the machine learning system 201 includes a random forest 209.

The machine learning system 201 may access data from the plurality of sources 207 in any suitable format including, for example, as summary tables (e.g., formatted as comma separated values) or in whole EMR (e.g., to be parsed by a script such as in Perl or SQL in the machine learning system 201). However the initial format, the data ultimately can be understood to include a plurality of entries 203. Each entry preferably includes a datum, or a value, that provides information to the system 201. The value may be a numerical value or it may be a string, such as a classification of disease code (e.g., ICD-9 code or ICD-10 code), which may be aggregated from different sources.

Most preferably, each entry 203 in the data is: specific to one patient from the population, and assigned to a pre-defined category. It will be understood that the data sources 207 may provide anonymized data. In such cases, each entry 203 is preferably specific to a patient and tracked to that patient by a patient ID value, which may be a random string or code. The external data sources 207 may provide the patient ID, or the machine learning system 201 may assign a patient ID to each entry 203. Each entry 203 preferably also has a category. For example, where a data entry 203 is an ICD-9 code, the category may be “ICD-9 Code” (and the value for the entry 203 is the ICD-9 code). In another example, where a data source 207 is an RNA-Seq assay for expression levels, a data entry 203 may be categorized as an expression level for one specific RNA and the value may be the expression level of that RNA. In yet one other example, where a data entry 203 is a patient's weight, the category may be “weight” and the value may be a mass in pounds or kilograms. The machine learning system 201 access the plurality of data sources 207 and discovers associations therein.

Discovering an association may include observing, in a plurality of patients, co-occurrences of event categories significantly different from an expected number of co-occurrences. In certain embodiments of the invention, inputs into a machine learning algorithm are scaled or normalized to facilitate meaningful comparisons across categorically different input types. Scaling and normalization methods are included. Scaling is used to divide each individual's data by a number to achieve some goal e.g., so that the range of values for all data lies in some interval, such as [0,1].

Scaling details may include choices such as “none”, “centering”, “autoscaling”, “rangescaling”, “paretoscaling” (by default=“autoscaling”). A number of different scaling methods are provided: “none”: no scaling method is applied; “centering”: centers the mean to zero; “autoscaling”: centers the mean to zero and scales data by dividing each variable by the variance; “rangescaling”: centers the mean to zero and scales data by dividing each variable by the difference between the minimum and the maximum value; “paretoscaling”: centers the mean to zero and scales data by dividing each variable by the square root of the standard deviation. Unit scaling divides each variable by the standard deviation so that each variance is equal to 1.

Normalization details are included and may be used. As with scaling, normalization may be used to divide or shift the total dataset to, for example, facilitate comparison of data from unlike source or of unlike formatting. For example, one could use the z-score of the data points: (z−μ)/σ. This normalization is determined by the mean of the data and its variance.

A number of different normalization methods are provided: “none”: no normalization method is applied; “pqn”: Probabilistic Quotient Normalization is computed as described in Dieterle, 2006, Probabilistic quotient normalization as robust method to account for dilution of complex biological mixtures. Application in 1H NMR metabonomics, Anal Chem 78(13):4281-90, incorporated by reference; “sum”: samples are normalized to the sum of the absolute value of all variables for a given sample; “median”: samples are normalized to the median value of all variables for a given sample; “sqrt”: samples are normalized to the root of the sum of the squared value of all variables for a given sample.

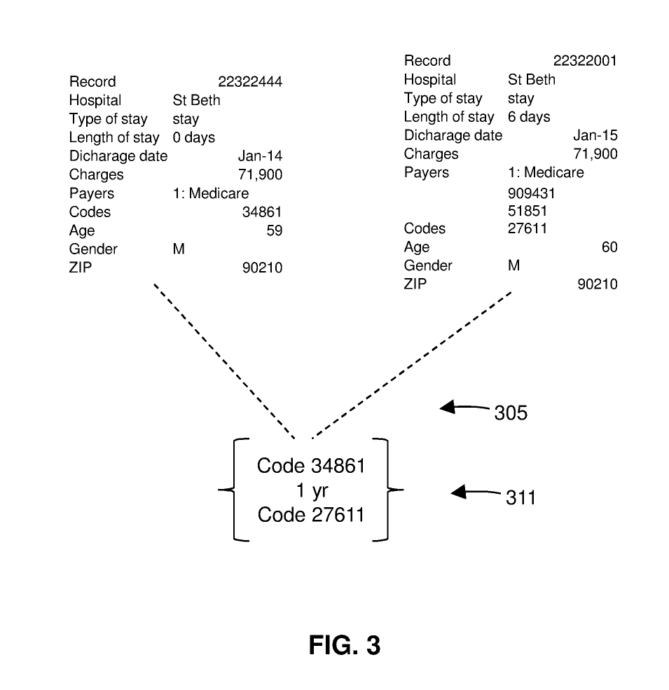

FIG. 3 shows one example of a machine learning system 201 discovering 115 associations in the data. In the depicted embodiment, the system has read 305 from two different medical records and observed the co-occurrence of two different diagnostic codes (34861 and 27611) within a 1 year span for a patient. The system 201 has observed this co-occurrence a number of times that is greater than the number that would be observed if those codes co-occurred within that time span only at random. The system creates an object 311 representing that the co-occurrence has been learned. Interestingly, that object 311—the association itself—can be added back into the data sources 207 as an entry 203 itself. Here, the system 201 will add the discovered 115 associations 311 into the data 207 as an event 203. Then, the system 201 may proceed and continue to discover 115 other associations in the data 207 that includes the initially-discovered association 311.

Systems and methods of the disclosure include a machine learning system 201. The machine learning system 201 is preferably implemented in a tangible, computer system built for implementing methods described herein.

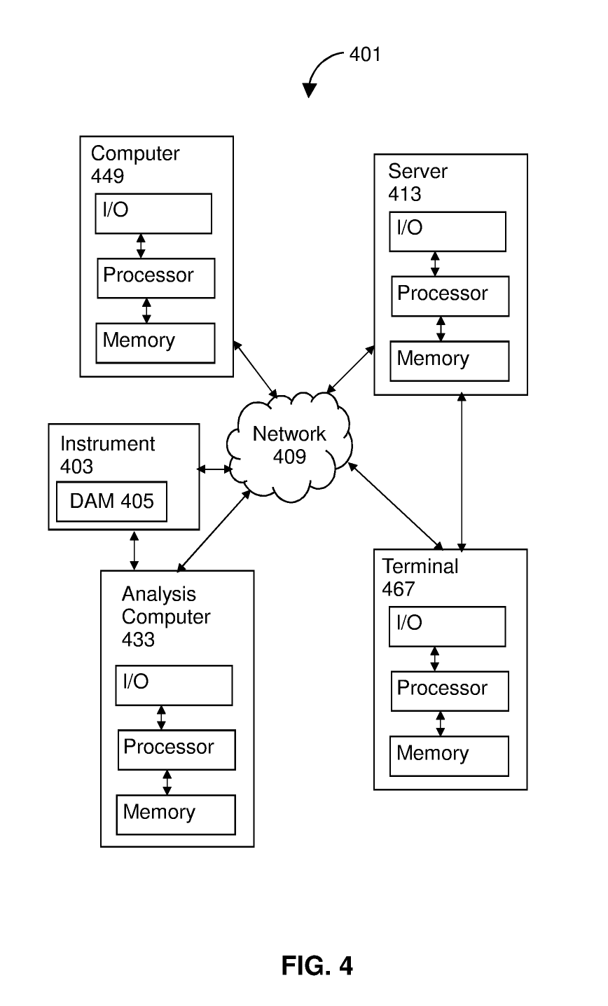

FIG. 4 diagrams a system 401 for predicting health status. The system 401 includes at least one computer 449, such as a laptop or desktop computer, than can be accessed by a user to initiate methods of the invention and obtain results. The system 401 preferably also includes at least one server sub-system 413 and either or both of the computer 449 and the server sub-system 413 may include and provide the machine learning system 201. The server subsystem 413 may have a dedicated terminal computer 467 for accessing the server sub-system 413. Additionally, in some embodiments, the system 401 operates in communication with a lab, such as a clinical services laboratory, which may include an analysis instrument 403 such as a nucleic acid sequencing instrument. The analysis instrument 403 may have its own data acquisition module 405, such as, for example, the flow cell and associated optical and electronic instruments of a nucleic acid sequencer such the sequencer sold under the trademark HISEQ or MISEQ by Illumina, Inc. The instrument 403 may have its own built-in or connected instrument computer 433. Any or all of the computer 449, server subsystem 413, terminal computer 467, instrument 403, and instrument computer 433 may exchange data over communications network 409, which may include elements of a local area network (LAN), a wide area network (WAN) the Internet, or combinations thereof. Each of computer 449, server subsystem 413, terminal computer 467, and instrument computer 433, when included, preferably includes at least one processor coupled to one or more input/output devices and a tangible, non-transitory memory subsystem. The I/O devices may include one or more of: monitor, keyboard, mouse, trackpad, touchpad, touchscreen, Wi-Fi card, cellular antenna, network interface cards, or others. The memory subsystem preferably includes one or more of RAM and a disc drive, such as a magnetic hard drive or solid state drive.

The system 401 contains instructions stored in the memory that are executable by one or more of processors to cause the system to discover, via the machine learning system 201, associations in data from a plurality of data sources 207 obtained from a population and correlate the associations to health status of patients in the population. Preferably, each entry 203 in the data is: specific to one patient from the population, and assigned to a pre-defined category. The system 401 discovers the associations by observing, in a plurality of patients, co-occurrences of event categories significantly different from an expected number of co-occurrences. In some embodiments, the system 401 is operable to add the discovered associations into the data as events and continue to discover associations in the data that includes the initially-discovered associations.

For the correlation 129 step, the machine learning system 201 may correlate to the associations to health statuses, such as known patient outcomes. The known patient outcomes provided to the machine learning algorithm may be, for example, a simple diagnosis (e.g., the patient was confirmed positive for a disease), a prognosis (i.e., good, fair or poor), treatment selection, mortality, comorbidity, disease severity, treatment compliance, know response to a treatment (i.e., effectiveness of treatment), and quality of life (e.g., changes in quality of live over the time span beginning at diagnosis). Depending on the outcomes provided to the machine learning algorithm, the trained algorithm can then be used to identify patterns indicative of the various outcomes and then to determine a likelihood of a patient having an outcome, or a combination of outcomes based on the claims data. Furthermore, the algorithm can differentiate between associations related to early-onset forms of a disease (e.g., juvenile forms) and late-onset forms of a disease (e.g., adult forms). In certain embodiments, the trained algorithm can then be used to further specify patterns indicative of the various outcomes and to determine a likelihood of a patient having an outcome based on the analysis of not only claims data, but on other data sets described herein. In one or more embodiments, the data sets are comprised of claims data and geographic data. In certain embodiments, the trained algorithm can be used to specify patterns in the data sets indicative of the various outcomes of a specific geography. In yet another embodiment, data from one geography are compared to that of a different geography to identify an increased likelihood of a patient having a disease in one of the geographies. When comparing the geographies, both geographies have a statically significant number of similarities and one of the geographies has limited outcome data for a disease. Identifying an increased likelihood of a patient having a disease in the geography with limited outcome data, is made by assessing the data that has been identified as being statistically significant.

Where the algorithm is trained on treatment outcomes, it can then be used to predict a patient's responsiveness to various disease specific therapies. Accordingly, methods may include recommending a treatment based in part on the prediction where a certain treatment will only be recommended for patients likely to respond thereto. In certain embodiments, the recommended treatment may be provided in a report for the patient or a treating physician. In other embodiments, based on the severity of the disease, the algorithm may also provide a cost projection for treatment. In some embodiments, the treatment may be prescribed for the patient or administered to the patient.

The method 101 and system 401 may be provided with patient data from an individual. That is, the machine learning system 201 has learned, from the plurality of data sources 207, patterns or associations that are predictive of disease. The system 201 may then be applied to an individual to predicting a health state for the individual when the patient data presents one or more of the discovered associations. The predicted health state may be in any suitable format. For example, the predicted health state is presented as a form of future diagnosis. The machine learning system alerts a health professional that the individual patient is presenting results that are most consistent with a diagnosis, within a certain time frame in the future, of a specific disease.

An advantage of the present invention is the ability to identify at-risk patients before the onset of a disease. Once patients having an increased risk of developing a disease are identified, they may be subjected to more frequent screening or additional testing for the disease so that development of the disease can be caught early and treated quickly. In certain embodiments, a patient identified as being at increased risk of developing a disease may receive preventative treatments targeted at preventing or delaying the development of the disease or of symptoms of the disease.

Methods employed via the machine learning system may have particular usefulness and sensitivity in detecting hallmarks of the future onset of certain degenerative diseases including, for example, multiple sclerosis, irritable bowel syndrome, Crohn's disease, ulcerative colitis, amyotrophic lateral sclerosis, fibromyalgia, rheumatoid arthritis, or lupus. The system 401 may use that information to provide a report, for use by a health professional in counseling or treating a patient.

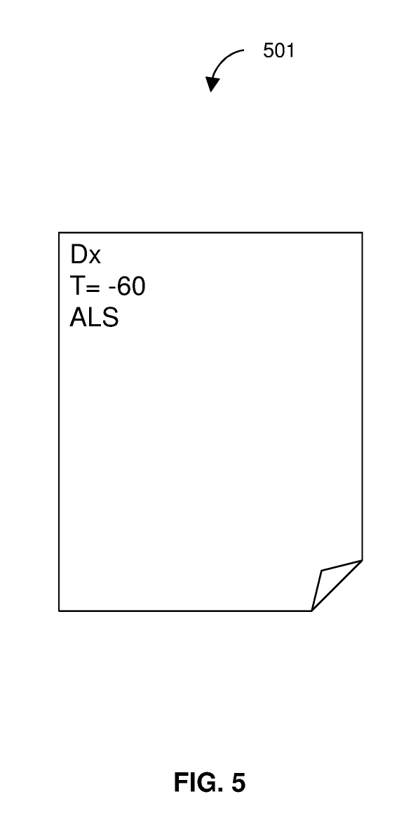

FIG. 5 shows a report 501 with a prediction. A report 501 may take any suitable format. For example, in certain embodiments, the report is an electronic document that is both human-readable and machine-readable, such as a PDF with text-searchable fields or an XML document shared within a system that applies style sheets for display. The report 501 may include information identifying a patient, a disease, and an onset. The onset may be prediction or future time. For example, the report may predict that an individual is at risk for a diagnosis, in 60 months, of amyotrophic lateral sclerosis. In certain embodiments, the methods and systems are operated by a clinical services laboratory that performs an assay on a sample from the patient and also operates the machine learning system to discover associations (e.g., between the assay results and claims data) that are predictive of a future disease state, such as diagnosis, comorbidity, severity, treatment compliance, reoccurrence of disease, or prognosis of disease. In such embodiments, the clinical services laboratory provides the report 501 to a health professional such as the patient's primary care provider.

In an illustrative example, a lab performs the method 501 in a way that includes receiving a sample from the individual, performing an assay on the sample to produce clinical results, and including the clinical results in the patient data from the individual. For example, a blood or plasma sample may be sent to the lab by overnight mail. The lab extracts nucleic acid from the sample and sequences the nucleic acid. The lab may receive the blood or plasma in a collection tube such as a blood collection tube sold under the trademark VACUTAINER by BD, extract nucleic acid from the sample using a commercially-available kit, perform library preparation and sequencing such as RNA-Seq using, for example, an Illumina sequencing instrument. The lab thereby obtains results that include sequences or expression level. The lab provides the results along with claims data or clinical diagnostic codes from the individual to the machine learning system and returns, to the doctor, a report 501 with a prediction of a future disease state.

As discussed above, the plurality of data sources 207 may include a variety of types of clinical data. Certain embodiments of the methods also combine, with electronic clinical data, biological data or test results (e.g., by performing an assay to generate the results). Those assay results may be from sequencing and thus may include genomic information from the patient.

In some embodiments, genomic information is obtained from a biological sample of a patient. Genetic data can be obtained, for example, by conducting an assay on a sample from a male or female that identifies variants present within DNA. The presence of certain SNPs in certain genetic regions or abnormal expression levels of those genetic regions may be indicative of a disease outcome, or may contribute to the outcome. Exemplary variants include, but are not limited to, a single nucleotide polymorphism, a single nucleotide variant, a deletion, an insertion, an inversion, a genetic rearrangement, a copy number variation, chromosomal microdeletion, genetic mosaicism, karyotype abnormality or a combination thereof. Methods of detecting variations (e.g., mutations) are known in the art. Methods of performing whole genome sequencing are known in the art. A sample may include a human tissue or bodily fluid and may be collected in any clinically acceptable manner. A tissue is a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS. A sample also may be media containing cells or biological material. A sample may also be a blood clot, for example, a blood clot that has been obtained from whole blood after the serum has been removed. In certain embodiments, the sample is blood, saliva, or semen collected from the subject. Genetic information from the sample can be obtained by nucleic acid extraction from the sample. Methods for extracting nucleic acid from a sample are known in the art. See for example, Maniatis, et al., Molecular Cloning: A Laboratory Manual, Cold Spring Harbor, N.Y., pp. 280-281, 1982, the contents of which are incorporated by reference herein in their entirety.

Those assay results are combined into the plurality of data sources 207. Preferably, the plurality of data sources comprises one or more of claims data, demographic data, geographic data, medical history, genetic data, and laboratory test results. In a most preferred embodiment, the data sources include claims data. Insurance claim data may include Healthcare Common Procedures Coding System (HCPCS), Current Procedural Terminology (CPT), or International Classification of Diseases (ICD) Clinical Modifications (CM), National Drug Codes (NDCs), International Classification of Primary Care (ICPC), or International Classification of Functioning, Disability and Health (ICF) codes for example. Insurance claim data may include, for example, individual level patient diagnoses, procedures, prescribed therapies, symptoms, geographic location, demographic information, and/or provider information and can be provided with associated chronological data. Claims data can be provided by medical providers or insurers for analysis. In other embodiments, methods of the invention also use phenotypic data, environmental data, demographic data, geographic data, genetic data, clinical data, treatment data and insurance claim data to determine unique patterns or signatures associated with specific diseases. These data can be derived from a variety of publically available sources, such as PubMed, government databases, for example those databases available on Data.gov. By comparing claims data of healthy patients to claims data of diseased patients or to known outcomes, one can identify patterns in the data that are indicative of certain diseases or disease outcomes. In certain embodiments, the claims data and associated known outcomes may be subjected to machine learning analysis to identify patterns most predictive of disease. In other embodiments, other data sets, such as phenotypic data, environmental data, demographic data, geographic data, genetic data, clinical data and treatment data are also subjected to machine learning analysis to further identify patterns most predicative of disease. All datasets may be subjected to machine learning analysis simultaneously, alternatively, datasets maybe analyzed individually, or in a layered approach to further refine the patterns identified as predictive of disease.

Any machine learning algorithm may be used to analyze the data including, for example, a random forest, a support vector machine (SVM), or a boosting algorithm (e.g., adaptive boosting (AdaBoost), gradient boost method (GBM), or extreme gradient boost methods (XGBoost)), or neural networks such as H2O.

Machine learning algorithms generally are of one of the following types: (1) bagging (decrease variance), (2) boosting (decrease bias), or (3) stacking (improving predictive force). In bagging, multiple prediction models (generally of the same type) are constructed from subsets of classification data (classes and features) and then combined into a single classifier. Random Forest classifiers are of this type. In boosting, an initial prediction model is iteratively improved by examining prediction errors. AdaBoost and eXtreme Gradient Boosting are of this type. In stacking models, multiple prediction models (generally of different types) are combined to form the final classifier. These methods are called ensemble methods. The fundamental or starting methods in the ensemble methods are often decision trees. Decision trees are non-parametric supervised learning methods that use simple decision rules to infer the classification from the features in the data. They have some advantages in that they are simple to understand and can be visualized as a tree starting at the root (usually a single node) and repeatedly branch to the leaves (multiple nodes) that are associated with the classification.

In a preferred embodiment, methods 101 and systems 401 of the invention use a machine learning system 201 that uses a random forest 209. Random forests use decision tree learning, where a model is built that predicts the value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable.

SVMs can be used for classification and regression. When used for classification of new data into one of two categories, such as having a disease or not having a disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering. See Ben-Hur, 2001, Support Vector Clustering, J Mach Learning Res 2:125-137, incorporated by reference.

Boosting algorithms are machine learning ensemble meta-algorithms for reducing bias and variance. Boosting is focused on turning weak learners into strong learners where a weak learner is defined to be a classifier which is only slightly correlated with the true classification while a strong learner is a classifier that is well-correlated with the true classification. Boosting algorithms consist of iteratively learning weak classifiers with respect to a distribution and adding them to a final strong classifier. The added classifiers are typically weighted in based on their accuracy. Boosting algorithms include AdaBoost, gradient boosting, and XGBoost. See Freund, 1997, A decision-theoretic generalization of on-line learning and an application to boosting, J Comp Sys Sci 55:119; and Chen, 2016, XGBoost: A Scalable Tree Boosting System, arXiv:1603.02754, both incorporated by reference.

Neural networks, modeled on the human brain, allow for processing of information and machine learning. Neural networks include nodes that mimic the function of individual neurons, and the nodes are organized into layers. Neural networks include an input layer, an output layer, and one or more hidden layers that define connections from the input layer to the output layer. Systems and methods of the invention may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015), each of the aforementioned references are incorporated by reference.

Deep learning neural networks (also known as deep structured learning, hierarchical learning or deep machine learning) include a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower level features to form a hierarchical representation. Those features are preferably represented within nodes as feature vectors. Deep learning by the neural network includes learning multiple levels of representations that correspond to different levels of abstraction; the levels form a hierarchy of concepts. In some embodiments, the neural network includes at least 5 and preferably more than ten hidden layers. The many layers between the input and the output allow the system to operate via multiple processing layers.

Deep learning is part of a broader family of machine learning methods based on learning representations of data. An observation can be represented in many ways such as a vector of intensity values per pixel, or in a more abstract way as a set of edges, regions of particular shape, etc. Those features are represented at nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

Within the network, nodes are connected in layers, and signals travel from the input layer to the output layer. In certain embodiments, each node in the input layer corresponds to a respective one of the features from the training data. The nodes of the hidden layer are calculated as a function of a bias term and a weighted sum of the nodes of the input layer, where a respective weight is assigned to each connection between a node of the input layer and a node in the hidden layer. The bias term and the weights between the input layer and the hidden layer are learned autonomously in the training of the neural network. The network may include thousands or millions of nodes and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights, and is sometimes done to train the network using known correct outputs. See WO 2016/182551, U.S. Pub. 2016/0174902, U.S. Pat. No. 8,639,043, and U.S. Pub. 2017/0053398, each incorporated by reference.

In some embodiments, the datasets are used to cluster a training set. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node.

Regression analysis is a statistical process for estimating the relationships among variables such as features and outcomes. It includes techniques for modeling and analyzing relationships between a multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Any suitable machine learning algorithm may be included. In preferred embodiments, the machine learning system 201 includes a random forest 209. The machine learning system may learn in a supervised or unsupervised fashion. A machine learning system that learns in an unsupervised fashion may be referred to as an autonomous machine learning system. While other versions are within the scope of the invention, an autonomous machine learning system can employ periods of both supervised and unsupervised learning. The random forest 209 may be operated autonomously and may include periods of both supervised and unsupervised learning. See Criminisi, 2012, Decision Forests: A unified framework for classification, regression, density estimation, manifold learning and semi-supervised learning, Foundations and Trends in Computer Graphics and Vision 7(2-3):81-227, incorporated by reference. Thus in some embodiments, the autonomous machine learning system 201 comprises a random forest 209. In some embodiments, the autonomous machine learning system 201 discovers the associations via operations that include at least a period of unsupervised learning. In preferred embodiments, the discovered associations including patterns of association between claims data and at least one other data source such as RNA expression levels.

The invention relates to methods and systems for identifying disease based on the analysis of datasets. Datasets are comprised of data from a plurality of normal patients and diseased patients with known outcomes. Importantly, insurance claims data provide a wealth of patient information that can be mined for patterns indicative of disease. By training machine learning algorithms on data from a plurality of patients with known disease outcomes, those patterns can be identified and then used to identify outcomes that are characteristic of a disease of interest. Trained machine learning algorithms can then quickly identify disease patients with specific, potentially hard to diagnose diseases by combing the mass amounts of data generated every day across the world by comparing a patient's specific attributes to the features of a selected disease. The algorithms can differentiate between associations related to early-onset forms of a disease (e.g., juvenile forms) and late-onset forms of a disease (e.g., adult forms) to allow for efficient diagnosis. The algorithms may be used to identify increased risk of disease prior to onset, possibility of comorbidity, identify prognosis, severity, or even predict treatment response or compliance. Further, the algorithms can identify misdiagnosed patients, saving time and money in their treatment. By providing accurate and early diagnoses of disease, methods of the invention allow for earlier and more efficient treatment of the disease, prolonging life expectancies, increasing patients' quality of life, and avoiding unnecessary or harmful treatment.

By further training machine learning algorithms on data with and without outcomes, patterns are identified in those data without outcomes which are associated with a disease of interest. The methods disclosed herein involve selecting known features that are characteristic of the disease of interest so as to filter out data not associated with the disease of interest. The methods further involve inputting a patient's specific attributes and comparing the attributes to the remaining disease data to provide a disease outcome for the patient. In some embodiments, the method includes identifying a score based on the comparison, and the outcome of the disease is identified based on the score.

To accomplish this, datasets are obtained from publicly available data sources and proprietary data sources and contain population data such as outcome data, phenotypic data, environmental data, demographic data, geographic data, genetic data, clinical data, insurance claim data and treatment data. Any disease, including cancers, neurological diseases, inflammatory diseases, rheumatic diseases, and autoimmune diseases may be examined using methods of the invention. In various embodiments, methods of the invention provide for diagnosis of diseases such as multiple sclerosis (MS), Parkinson's disease, atherosclerosis, stroke, asthma, uveitis, sinusitis, angioedema, psoriasis, psoriatic arthritis, multiple sclerosis, Alzheimer's disease, fibromyalgia, rheumatoid arthritis, lupus, ankylosing spondylitis, Hashimoto's thyroiditis, Sjögren's syndrome, Graves' disease, inflammatory bowel disease, Crohn's disease, ulcerative colitis, celiac disease, pernicious anemia, sinusitis, epilepsy and Parkinson's disease, through analysis of data sets, and data sets specifically including insurance claims data. Insurance claims data, unlike biopsies or blood draws, is generated by default as a byproduct of medical interactions. Accordingly, general screens of patient insurance claim data can be implemented without physically affecting the patients or requiring any action on their part.

Diseases each have their own set of features that are characteristic of the particular disease. Features of a disease can be symptoms and signs associated with a particular system of the body, such as neurological, musculoskeletal, ocular, gastrointestinal, cardiovascular, urinary, reproductive, pulmonary, endocrine, and integumentary systems. Features associated with a disease can also include psychological characteristics, speech and voice alterations, genetic biomarkers, laboratory tests, medical procedures, and interventions. Often times features of one disease overlap with that of another disease, or are masked by another feature, leading to misdiagnosis or late diagnosis, and therefore delayed treatment. Additionally, symptoms and signs associated with a disease might be nonspecific and variable depending on the patient, and there is no one test to particularly identify the disease because of the nonspecific and variable features. Specifically, in autoimmune and inflammatory diseases, symptoms and signs of diseases in are very similar with slight differences, or hard to identify differences. Additionally, diseases have various states, such as severity, prognosis, reoccurrence and comorbidity, and patients respond differently to treatment and comply with treatment protocols differently. Accordingly, the machine learning algorithm is trained on this data. Methods include providing patient data from an individual; and predicting, by the machine learning system 201, a health state for the individual when the patient data presents one or more of the discovered associations.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of predicting health status, the method comprising:
determining, via an autonomous machine learning system, associations in data from a plurality of data sources obtained from a population; and
correlating the associations to health status of patients in the population.

2. The method of claim 1, wherein each entry in the data is specific to one patient from the population, and assigned to a pre-defined category.

3. The method of claim 2, wherein discovering an association includes observing, in a plurality of patients, co-occurrences of event categories significantly different from an expected number of co-occurrences.

4. The method of claim 2, further comprising:
adding the discovered associations into the data as events; and
continuing to discover associations in the data that includes the initially-discovered associations.

5. The method of claim 1, further comprising:
providing patient data from an individual; and
predicting, by the autonomous machine learning system, a health state for the individual when the patient data presents one or more of the discovered associations.

6. The method of claim 5, further comprising:
receiving a sample from the individual;
performing an assay on the sample to produce clinical results; and
including the clinical results in the patient data from the individual.

7. The method of claim 6, wherein the sample comprises nucleic acid from the individual and the assay includes sequencing the nucleic acid, wherein the clinical results include sequences or expression level, and further wherein providing the patient data includes obtaining clinical diagnostic codes from the individual.

8. The method of claim 1, wherein the plurality of data sources comprise one or more of claims data, demographic data, geographic data, medical history, genetic data, and laboratory test results.

9. The method of claim 1, wherein the autonomous machine learning system comprises a random forest.

10. The method of claim 1, wherein the autonomous machine learning system discovers the associations via operations that include at least a period of unsupervised learning.

11. The method of claim 1, wherein the discovered associations including patterns of association between claims data and at least one other data source.

12. The method of claim 11, wherein the at least one other data source includes genomic data.

13. The method of claim 12, wherein the genomic data is RNA expression data.

14. The method of claim 12, further comprising:
providing patient data from an individual; and
predicting, by the machine learning system, a health state for the individual when the patient data presents one or more of the discovered associations.

15. The method of claim 14, wherein the patient data presents a discovered association between claims data and genomic data, and further wherein the predicted health state for the individual includes a predicted onset of a disease.

16. The method of claim 14, wherein the disease is selected from the group comprising atherosclerosis, depression, migraine, cancer, chronic obstructive pulmonary disease (COPD), congestive heart failure (CHF), type 1 diabetes, type 2 diabetes, stroke, asthma, uveitis, sinusitis, angioedema, psoriasis, psoriatic arthritis, multiple sclerosis, Alzheimer's disease, dementia, Parkinson's disease, posttraumatic stress disorder (PTSD), fibromyalgia, rheumatoid arthritis, lupus, ankylosing spondylitis, Hashimoto's thyroiditis, Sjögren's syndrome, Graves' disease, irritable bowel syndrome, inflammatory bowel disease, Crohn's disease, ulcerative colitis, celiac disease, pernicious anemia, and sinusitis.

17. The method of claim 16, wherein the autonomous machine learning system comprises one selected from the group consisting of a random forest, a support vector machine, and a neural network.

18. The method of claim 1, wherein the discovered associations include a patient-specific pattern occurring within claims data, and wherein a recurrence of the patient-specific pattern within the claims data is correlated to a later onset of a disease.

19. The method of claim 18, wherein the disease is an autoimmune, inflammatory or neurodegenerative disease.

20. The method of claim 18, wherein the patient-specific pattern includes combinations of ICD-9 codes reported over time that are predictive of the disease.

21. The method of claim 1, wherein the autonomous machine learning system comprises one selected from the group consisting of a random forest, a support vector machine, and a neural network.

22. The method of claim 1, wherein the health status comprises one selected from the group consisting of a disease diagnosis, comorbidity of disease, severity of a disease, treatment compliance, reoccurrence of disease, and prognosis of disease.

23. A method for identifying co-morbidities in a patient, the method comprising the steps
determining, via an autonomous machine learning system, associations in data from a plurality of data sources obtained from a population; and
correlating the associations to health status of patients in the population in order to identify comorbidities for the patient.
